(19) United States
(12) Patent Application Publication
(10) Pub. No.: US 2008/0287502 A1
(43) Pub. Date: Nov. 20, 2008

(54) Transdermal Administration of Proton Pump Inhibitors

(75) Inventors: Tsung-Min Hsu, Minneapolis, MN (US); Eric C. Jacobson, San Diego, CA (US); Alan T.J. Hickey, Eden Prairie, MN (US); Nicole T. Gricenko, St. Louis Park, MN (US); Eric C. Luo, Plano, TX (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(73) Assignee: DERMATRENDS, INC., San Diego, CA (US)

(21) Appl. No.: 11/547,335

(22) PCT Filed: Mar. 30, 2005
(86) PCT No.: PCT/US2005/010813

§ 371 Date: Jul. 30, 2008

Related U.S. Application Data
(60) Provisional application No. 60/557,790, filed on Mar. 30, 2004.

(51) Int. Cl. A61K 31/4439; A61K 47/10; A61P 43/00

(52) U.S. Cl. 514/338; 514/772

(57) ABSTRACT

A method and composition for the transdermal administration of proton pump inhibitors such as substituted pyridyl methylsulfinyl benzimidazoles, and in particular, omeprazole, lansoprazole, esomeprazole, pantoprazole and raberprazole. The method and composition include the use of a hydroxide-releasing agent as a permeation enhancer to increase the flux of the protein pump inhibitor through a patient's skin or mucosal tissues and optionally also include the use of a carrier such as 1,3-butanediol, dipropylene glycol, and hexylene glycol.

### TECHNICAL FIELD

This invention relates generally to the topical and transdermal administration of pharmacologically active agents, and more particularly relates to methods and compositions for enhancing the permeability of skin or mucosal tissue to topically applied pharmacologically active agents. The invention also relates to formulation and use of proton pump inhibitors, and particularly, substituted pyridyl methylsulfinyl benzimidazoles.

### BACKGROUND ART

The delivery of drugs through the skin provides many advantages; primarily, such a means of delivery is a comfortable, convenient and noninvasive way of administering drugs. The variable rates of absorption and metabolism encountered in oral treatment are avoided, and other inherent inconveniences—e.g., gastrointestinal irritation and the like—are eliminated as well. Transdermal drug delivery also makes possible a high degree of control over blood concentrations of any particular drug.

Skin is a structurally complex, relatively thick membrane. Molecules moving from the environment into and through intact skin must first penetrate the stratum corneum and any material on its surface. They must then penetrate the viable epidermis, the papillary dermis, and the capillary walls into the blood stream or lymph channels. To be so absorbed, molecules must overcome a different resistance to penetration in each type of tissue. Transport across the skin membrane is thus a complex phenomenon. However, it is the cells of the stratum corneum which present the primary barrier to absorption of topical compositions or transdermally administered drugs. The stratum corneum is a thin layer of dense, highly keratinized cells approximately 10-15 microns thick over most of the body. It is believed to be the high degree of keratinization within these cells as well as their dense packing which creates in most cases a substantially impermeable barrier to drug penetration. With many drugs, the rate of permeation through the skin is extremely low without the use of some means to enhance the permeability of the skin.

In order to increase the rate at which a drug penetrates through the skin, then, various approaches have been followed, each of which involves the use of either a chemical penetration enhancer or a physical penetration enhancer. Physical enhancement of skin permeation include, for example, electrophoretic techniques such as iontophoresis. The use of ultrasound (or “phonophoresis) as a physical penetration enhancer has also been researched. Chemical enhancers are compounds that are administered along with the drug (or in some cases the skin may be pretreated with a chemical enhancer) in order to increase the permeability of the stratum corneum, and thereby provide for enhanced penetration of the drug through the skin. Ideally, such chemical penetration enhancers (or “permeation enhancers,” as the compounds are referred to herein) are compounds that innocuous and serve merely to facilitate diffusion of the drug through the stratum corneum.

Various compounds for enhancing the permeability of skin are known in the art and described in the pertinent texts and literature. Compounds that have been used to enhance skin permeability include: sulfoxides such as dimethylsulfoxide (DMSO) and decylmethylsulfoxide (C.sub.10 MSO); ethers such as diethylene glycol monoethyl ether (available commercially as Transcutol™) and diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80) and lecithin (U.S. Pat. No. 4,783,450); the 1-substituted azacycloheptan-2-ones, particularly 1-n-dodecylcyclazacycloheptan-2-one (available under the trademark Azone M from Nelson Research & Development Co., Irvine, Calif.; see U.S. Pat. Nos. 3,989,816, 4,316,893, 4,405,616 and 4,557,934); alcohols such as ethanol, propanol, octanol, benzyl alcohol, and the like; fatty acids such as lauric acid, oleic acid and valeric acid; fatty acid esters such as isopropyl myristate, isopropyl palmitate, methylpropionate, and ethyl oleate; polyols and esters thereof such as propylene glycol, ethylene glycol, glycerol, butanediol, polyethylene glycol, and polyethylene glycol monolaurate (PEGML; see, e.g., U.S. Pat. No. 4,568,343); amides and other nitrogenous compounds such as urea, dimethylacetamide (DMA), dimethylformamide (DMF), 2-pyrrolidone, 1-methyl-2-pyrrolidone, ethanolamine, diethanolamine and triethanolamine; terpenes; alkanones; and organic acids, particularly salicylic acid and salicylates, citric acid and succinic acid. Percutaneous Penetration Enhancers, eds. Smith et al. (CRC Press, 1995) provides an excellent overview of the field and further background information on a number of chemical and physical enhancers.

Although many chemical permeation enhancers are known; there is an ongoing need for enhancers that are highly effective in increasing the rate at which a drug permeates the skin, do not result in skin damage, irritation, sensitization, or the like, and can be used to effect transdermal delivery of even high molecular weight drugs such as peptides, proteins, and nucleic acids. It has now been discovered that hydroxide-releasing agents are highly effective permeation enhancers, even when used without co-enhancers, provide all of the aforementioned advantages relative to known permeation enhancers. Furthermore, in contrast to conventional enhancers, transdermal administration of drugs with hydroxide-releasing agents as permeation enhancers, employed at the appropriate levels, does not result in systemic toxicity.

A particular class of compounds of particular interest includes substituted pyridyl methylsulfinyl benzimidazoles, also known as “proton pump inhibitor” (“PPI”). An early and isolated example of the attempted transdermal delivery of a PPI includes U.S. Pat. No. 5,124,158.

PPIs are highly acid labile and hence oral formulations are enteric-coated. Enteric coated formulations are expensive and time consuming to manufacture, and requires elaborate technology and equipment. Another disadvantage of enteric coated formulation is its moisture sensitivity.

WO94/25070 discloses oral composition containing a proton pump inhibitor in the form of enteric coated dry particles mixed with a dry gelling agent, the mixture may then be made into a paste-like gel prior to administration. The composition therefore requires enteric coating, with the afore-mentioned disadvantages associated with such formulation. Furthermore, because such a moist gel is not stable during long-term storage at room temperature it cannot be manufactured and sold as a ready-to-use formulation, rather it must be prepared ex tempore at the time of administration, making it inconvenient to use.

See also, Dentinger, et al., which describes the manner in which the PPI pantoprazole might be provided in a compounded oral liquid.

### BRIEF DESCRIPTION OF THE DRAWING

In the Drawing:

FIG. 1 shows a table of formulations used and described in the Examples.

FIG. 2 shows a graph showing human skin permeation of omeprazole from a matrix patch.

FIG. 3 shows a graph showing human skin permeation of omeprazole from a matrix patch (excluding cells B1 and D3).

FIGS. 4a and 4b show permeation data over time for various formulations as described in the Examples.

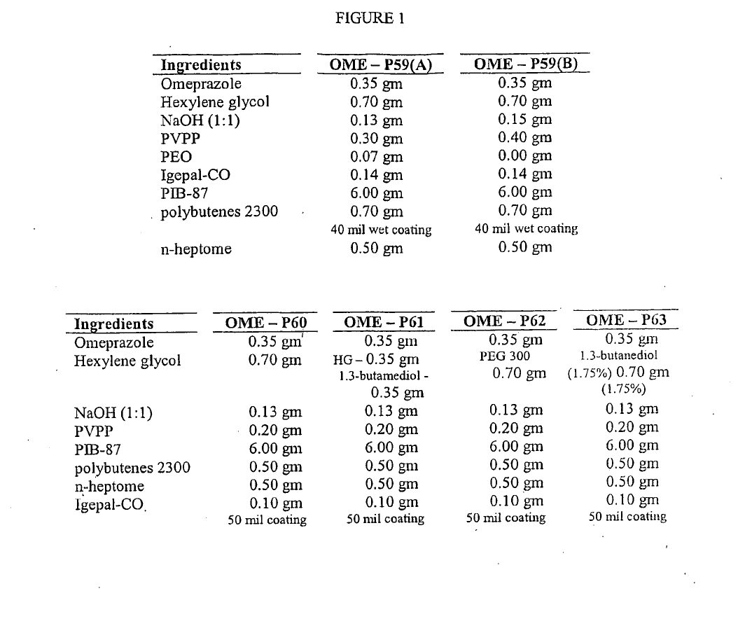

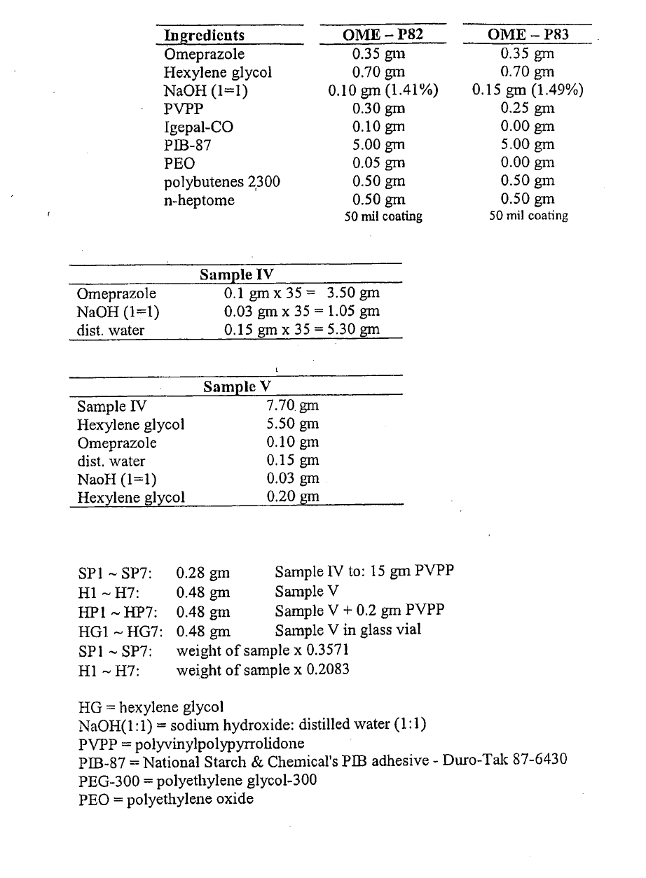

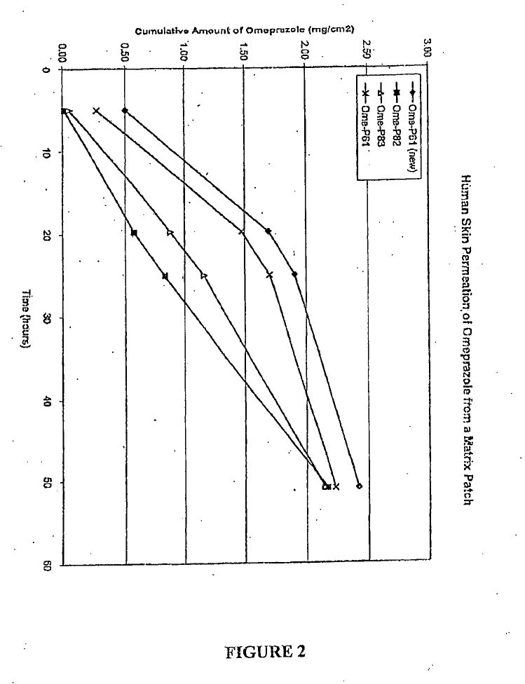

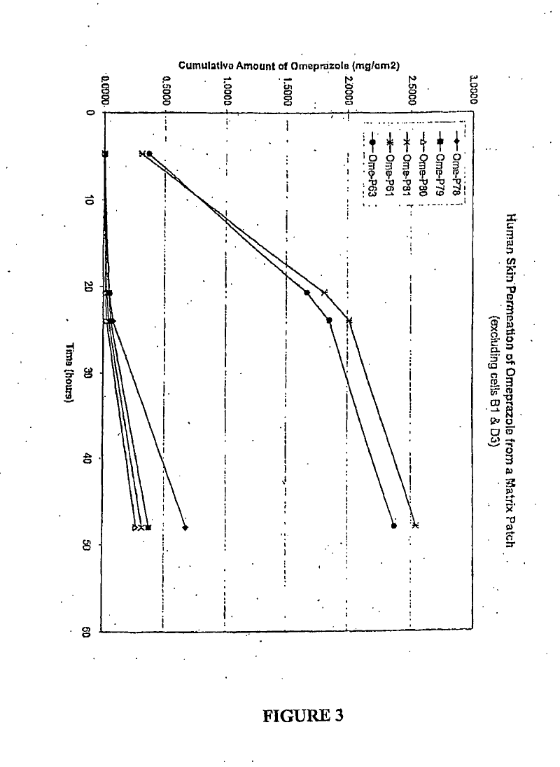

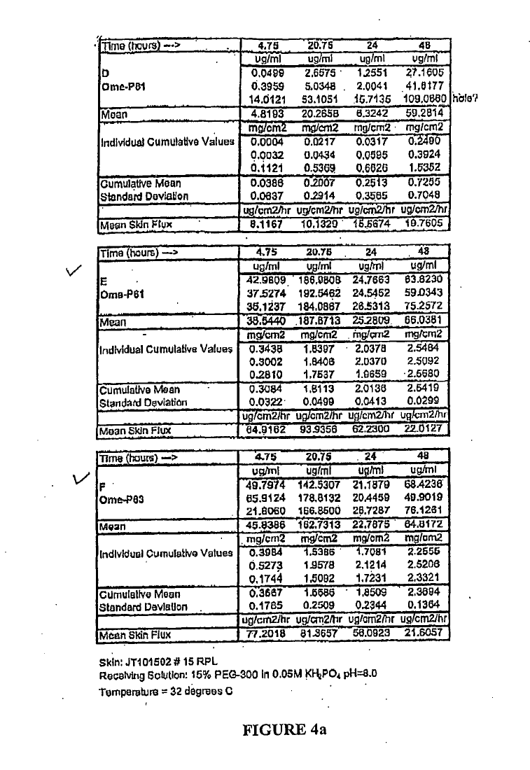

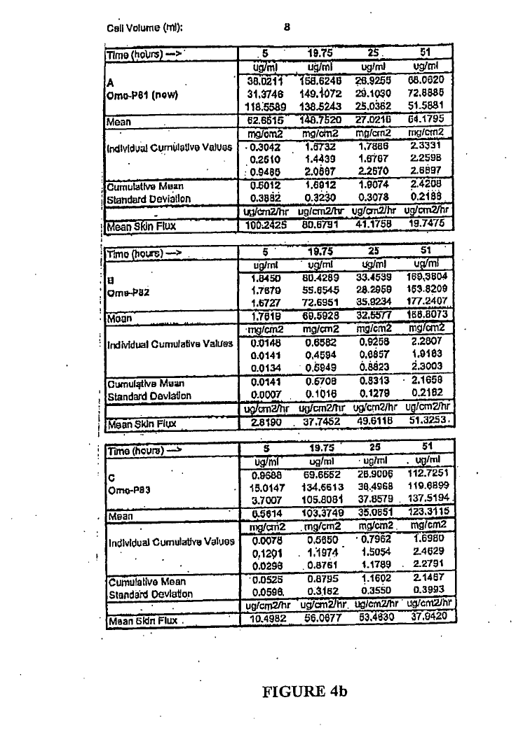

### SUMMARY OF THE INVENTION

The present invention provides a method for enhancing the rate at which an active agent comprising a PPI can be administered in stable form to a patient's body surface in order to permeate into and/or through the body surface. The invention provides a method wherein a hydroxide-releasing agent is employed as a permeation enhancer to increase the flux of the PPI through a patient's skin or mucosal tissue. The amount of hydroxide-releasing agent employed is optimized to enhance permeation while minimizing or eliminating the possibility of skin damage, irritation or sensitization. The hydroxide releasing agent and PPI are used in combination with a carrier such as an alkyl or alkylene diol or glycol, in order to both solubilize and stabilize the PPI for its intended use.

Surpisingly, the resulting composition provides an optimal combination of both PPI stability and permeation, in that it provides therapeutic levels to the body while meeting or exceeding the various stability and other requirements required by the FDA. In particular, preferred compositions meet or exceed the FDA guidelines for “Stability Testing of Drug Substances and Drug Products”, June 1998 and available at http://www.fda.gov/cder/guidance/1707dft.pdf, in that the compositions show no significant change during 6 months of storage under conditions of accelerated testing at 40 (+/−2) degrees C., 75% (+/−5%) relative humidity.

In a preferred embodiment, the PPI is a substituted pyridyl methylsulfinyl benzimidazole selected from the group consisting of omeprazole, lansoprazole, esomeprazole, pantoprazole and raberprazole, and is present at a concentration of between about 5 to about 30% by weight of the composition. Such compounds can be found in representative U.S. Pat. Nos. 4,508,905 and 5,708,017, the disclosures of which are incorporated herein by reference.

Such proton pump inhibitors are potent inhibitors of gastric acid secretion by inhibiting (H⁺K⁺)ATPase, the enzyme involved in the final step of hydrogen ion production in the parietal cells. Hence, PPI have been used in the treatment of gastric acid related diseases in humans. These diseases include gastric and duodenal ulcers. Peptic ulcers are common also in some animals, particularly in horses. Although the etiology of gastro-duodenal ulcers in horses has not been ascertained, it appears that stress plays an important roles in some cases.

In a further preferred embodiment, the alkyl or alkylene diol or glycol is selected from the group consisting of 1,3-butanediol, dipropylene glycol, and hexylene glycol, present in an amount of between about 5 to about 40% by weight, based on the weight of the composition.

### DETAILED DESCRIPTION

In one aspect of the invention, then, a method is provided for increasing the rate at which a PPI permeates through the body surface of a patient. The method involves administering the agent to a predetermined area of the patient's body surface in combination with a hydroxide-releasing agent in a predetermined amount effective to enhance the flux of the agent through the body surface without causing damage thereto. The predetermined amount of the hydroxide-releasing enhancer is preferably an amount effective to provide a pH at the body surface, i.e., during PPI administration, in the range of about 8 to 13, and preferably about 9 to about 11. If a skin patch is used, this is the preferred pH at the interface between the basal surface of the patch (i.e., the skin-contacting or mucosa-contacting surface of the patch) and the body surface. The optimal amount (or concentration) of any one hydroxide-releasing agent will, however, depend on the specific hydroxide-releasing agent, i.e., on the strength or weakness of the base, its molecular weight, and other factors as will be appreciated by those of ordinary skill in the art of transdermal drug delivery.

In a related aspect of the invention, a composition of matter is provided for delivering a PPI through a body surface using a hydroxide-releasing agent as a permeation enhancer. Generally, the formulation comprises (a) a therapeutically effective amount of a PPI, (b) a hydroxide-releasing agent in an amount effective to enhance the flux of the PPI through the body surface without causing damage thereto, and (c) a pharmaceutically acceptable carrier suitable for topical or transdermal PPI administration. The composition may be in any form suitable for application to the body surface, and may comprise, for example, a cream, lotion, solution, gel, ointment, paste or the like, and/or may be prepared so as to contain liposomes, micelles, and/or microspheres. The composition may be directly applied to the body surface or may involve use of a PPI delivery device. In either case, it is preferred although not essential that water be present in order for the hydroxide-releasing agent to generate hydroxide ions and thus enhance the flux of the PPI through the patient's body surface. Thus, a formulation or PPI reservoir may be aqueous, i.e., contain water, or may be nonaqueous and used in combination with an occlusive overlayer so that moisture evaporating from the body surface is maintained within the formulation or transdermal system during PPI administration.

In another aspect of the invention, a PPI delivery system is provided for the topical or transdermal administration of a PPI using a hydroxide-releasing agent as a permeation enhancer. The system will generally comprise: at least one PPI reservoir containing the PPI and the hydroxide-releasing agent in an amount effective to enhance the flux of the PPI through the body surface without causing damage thereto; a means for maintaining the system in PPI and enhancer transmitting relationship to the body surface; and a backing layer that serves as the outer surface of the device during use. The backing layer may be occlusive or nonocclusive, although it is preferably occlusive. The PPI reservoir may be comprised of a polymeric adhesive, which may serve as the basal surface of the system during use and thus function as the means for maintaining the system in PPI and enhancer transmitting relationship to the body surface. The PPI reservoir may also be comprised of a hydrogel, or it may be a sealed pouch within a “patch”-type structure wherein the PPI and hydroxide-releasing agent are present in the pouch as a liquid or semi-solid formulation.

It must be noted that, as used in this specification and the appended claims, the singular forms “a,” “an” and “the” include plural referents unless the context clearly dictates otherwise. Thus, for example, reference to “a pharmacologically active agent” includes a mixture of two or more such compounds, including at least one PPI, and reference to “a hydroxide-releasing agent” includes mixtures of two or more hydroxide-releasing agents, and the like.

In describing and claiming the present invention, the following terminology will be used in accordance with the definitions set out below.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. The present method of “treating” a patient, as the term is used herein, thus encompasses both prevention of a disorder in a predisposed individual and treatment of the disorder in a clinically symptomatic individual.

The term “hydroxide-releasing agent” as used herein is intended to mean an agent that releases free hydroxide ions in an aqueous environment. The agent may contain hydroxide ions and thus release the ions directly (e.g., an alkali metal hydroxide), or the agent may be on that is acted upon chemically in an aqueous environment to generate hydroxide ions (e.g., a metal carbonate).

The terms “active agent,” “drug” and “pharmacologically active agent” are used interchangeably herein to refer to a chemical material or compound that induces a desired effect, and include agents that are therapeutically effective, prophylactically effective, or cosmeceutically effective. Also included are derivatives and analogs of those compounds or classes of compounds specifically mentioned which also induce the desired effect. When used in describing a composition or method of the present invention, these terms refer to substituted pyridyl methylsulfinyl benzimidazoles. In a preferred embodiment, the present PPI is selected from the group consisting of omeprazole, lansoprazole, esomeprazole, pantoprazole and raberprazole.

By “therapeutically effective” amount is meant a nontoxic but sufficient amount of a PPI to provide the desired therapeutic effect.

By “transdermal” PPI delivery is meant administration of a drug to the skin surface of an individual so that the drug passes through the skin tissue and into the individual's blood stream, thereby providing a desired systemic and/or localized effect. The term “transdermal” is intended to include “transmucosal” drug administration, i.e., administration of a drug to the mucosal (e.g., sublingual, buccal, vaginal, rectal) surface of an individual so that the drug passes through the mucosal tissue and into the individual's blood stream. The term “body surface” is used to refer to skin or mucosal tissue.

By “predetermined area” of skin or mucosal tissue, which refers to the area of skin or mucosal tissue through which a drug-enhancer formulation is delivered, is intended a defined area of intact unbroken living skin or mucosal tissue. That area will usually be in the range of about 5 cm² to about 200 cm², more usually in the range of about 5 cm² to about 100 cm², preferably in the range of about 20 cm² to about 60 cm². However, it will be appreciated by those skilled in the art of drug delivery that the area of skin or mucosal tissue through which drug is administered may vary significantly, depending on patch configuration, dose, and the like.

“Penetration enhancement” or “permeation enhancement” as used herein relates to an increase in the permeability of the skin or mucosal tissue to the selected PPI, i.e., so that the rate at which the agent permeates therethrough (i.e., the “flux” of the agent through the body surface) is increased relative to the rate that would be obtained in the absence of permeation enhancement. The enhanced permeation effected through the use of such enhancers can be observed by measuring the rate of diffusion of drug through animal or human skin using method available to those skilled the art.

An “effective” amount of a permeation enhancer is meant a nontoxic, nondamaging but sufficient amount of the enhancer to provide the desired increase in skin permeability and, correspondingly, the desired depth of penetration, rate of administration, and amount of drug delivered.

“Carriers” or “vehicles” as used herein refer to carrier materials suitable for transdermal drug administration. Carriers and vehicles useful herein include any such materials known in the art which are nontoxic and does not interact with other components of the composition in a deleterious manner.

The term “aqueous” refers to a formulation or drug delivery system that contains water or that becomes water-containing following application to the skin or mucosal tissue.

Accordingly, the invention pertains to a method, composition and drug delivery system for increasing the rate at which a PPI permeates through the body surface of a patient, wherein the method involves administering the agent to a predetermined area of the patient's body surface in combination with a hydroxide-releasing agent in an amount effective to enhance the flux of the agent through the body surface without causing damage thereto.

The “hydroxide-releasing agent” is a chemical compound that releases free hydroxide ions in the presence of an aqueous fluid. The aqueous fluid may be natural moisture at the skin surface, or a patch or composition that is used may contain added water, and/or be used in connection with an occlusive backing. Similarly, any liquid or semisolid formulation that is used is preferably aqueous or used in conjunction with an overlayer of an occlusive material.

Any hydroxide-releasing agent may be used provided that the compound releases free hydroxide ions in the presence of an aqueous fluid. Examples of suitable hydroxide-releasing agents include, but are not limited to, inorganic hydroxides, inorganic oxides, and alkali metal or alkaline earth metal salts of weak acids. Inorganic hydroxides include, for example, ammonium hydroxide, alkali metal hydroxide and alkaline earth metal hydroxides, such as sodium hydroxide, calcium hydroxide, potassium hydroxide, magnesium hydroxide, and the like. Inorganic oxides include, for example, magnesium oxide, calcium oxide, and the like. Metal salts of weak acids include, for example, sodium acetate, sodium borate, sodium metaborate, sodium carbonate, sodium bicarbonate, sodium phosphate (tribasic), sodium phosphate (dibasic), potassium carbonate, potassium bicarbonate, potassium citrate, potassium acetate, potassium phosphate (dibasic), potassium phosphate (tribasic), ammonium phosphate (dibasic), and the like. Preferred hydroxide-releasing agents are metal hydroxides such as sodium hydroxide and potassium hydroxide.

It is important that the amount of hydroxide-releasing agent in any patch or formulation is optimized so as to increase the flux of the drug through the body surface while minimizing any possibility of skin damage. In general, this means that the pH at the body surface in contact with a formulation or drug delivery system of the invention (i.e., the interface between the body surface and the formulation or delivery system) should be in the range of approximately 8.0 to 13, preferably about 8.0 to 11.5, more preferably about 8.5 to 11.5. This will typically although not necessarily mean that the pH of the formulation or the drug composition contained within a delivery system will be in the range of approximately 8.0 to 13, preferably about 8.0 to 11.5, more preferably about 8.5 to 11.5.

For inorganic hydroxides, the amount of hydroxide-releasing agent will typically represent about 0.5 wt. % to about 5 wt. %, preferably about 0.5 wt. % to about 3 wt. %, more preferably about 0.75 wt. % to about 2 wt. % and optimally about 1 wt. %, of a topically applied formulation or of a drug reservoir of a drug delivery system, or “patch.”

Still greater amounts of hydroxide-releasing agent may be used by controlling the rate and/or quantity of release of the hydroxide-releasing agent preferably during the drug delivery period itself.

However, for all hydroxide-releasing agents herein, the optimum amount of any particular agent will depend on the strength or weakness of the base, the molecular weight of the base, and other factors such as the number of ionizable sites in the drug administered and any other acidic species in the formulation or patch. One skilled in the art may readily determine the optimum amount for any particular agent by ensuring that a formulation or drug delivery system

The PPI administered can be in any form suitable for topical, transdermal or transmucosal delivery and induces a desired local or systemic effect.

The amount of PPI administered will depend on a number of factors and will vary from subject to subject and depend on the particular drug administered, the particular disorder or condition being treated, the severity of the symptoms, the subject's age, weight and general condition, and the judgment of the prescribing physician. Other factors, specific to transdermal drug delivery, include the solubility and permeability of the carrier and adhesive layer in a drug delivery device, if one is used, and the period of time for which such a device will be fixed to the skin or other body surface. The minimum amount of drug is determined by the requirement that sufficient quantities of drug must be present in a device or composition to maintain the desired rate of release over the given period of application. The maximum amount for safety purposes is determined by the requirement that the quantity of drug present cannot exceed a rate of release that reaches toxic levels. Generally, the maximum concentration is determined by the amount of agent that can be received in the carrier without producing adverse histological effects such as irritation, an unacceptably high initial pulse of agent into the body, or adverse effects on the characteristics of the delivery device such as the loss of tackiness, viscosity, or deterioration of other properties.

The method of delivery of the active agent may vary, but necessarily involves application of a formulation or drug delivery system containing a hydroxide-releasing agent to a predetermined area of the skin or other tissue for a period of time sufficient to provide the desired local or systemic effect. The method may involve direct application of the composition as an ointment, gel, cream, or the like, or may involve use of a drug delivery device. In either case, Water must be present in order for the hydroxide-releasing agent to generate hydroxide ions and thus enhance the flux of the active agent through the patient's body surface. Thus, a formulation or drug reservoir may be aqueous, i.e., contain water, or may be nonaqueous and used in combination with an occlusive overlayer so that moisture evaporating from the body surface is maintained within the formulation or transdermal system during drug administration. In some cases, however, e.g., with an occlusive gel, a nonaqueous formulation may be used with or without an occlusive layer.

Suitable formulations include ointments, creams, gels, lotions, pastes, and the like. Ointments, as is well known in the art of pharmaceutical formulation, are semisolid preparations that are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington: The Science and Practice of Pharmacy, 19th Ed. (Easton, Pa.: Mack Publishing Co., 1995), at pages 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight; again, see Remington: The Science and Practice of Pharmacy for further information.

Creams, as also well known in the art, are viscous liquids or semisolid emulsions, either oil-in-water or water-in-oil. Cream bases are water-washable, and contain an oil phase, an emulsifier and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

As will be appreciated by those working in the field of pharmaceutical formulation, gels are semisolid, suspension-type systems. Single-phase gels contain organic macromolecules distributed substantially uniformly throughout the carrier liquid, which is typically aqueous, but also, preferably, contain an alcohol and, optionally, an oil. Preferred “organic macromolecules,” i.e., gelling agents, are crosslinked acrylic acid polymers such as the “carbomer” family of polymers, e.g., carboxypolyalkylenes that may be obtained commercially under the Carbopol™ trademark. Also preferred are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Lotions, which are preferred for delivery of cosmetic agents, are preparations to be applied to the skin surface without friction, and are typically liquid or semiliquid preparations in which solid particles, including the active agent, are present in a water or alcohol base. Lotions are usually suspensions of solids, and preferably, for the present purpose, comprise a liquid oily emulsion of the oil-in-water type. Lotions are preferred formulations herein for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethyl-cellulose, or the like.

Pastes are semisolid dosage forms in which the active agent is suspended in a suitable base. Depending on the nature of the base, pastes are divided between fatty pastes or those made from a single-phase aqueous gels. The base in a fatty paste is generally petrolatum or hydrophilic petrolatum or the like. The pastes made from single-phase aqueous gels generally incorporate carboxymethylcellulose or the like as a base.

Formulations may also be prepared with liposomes, micelles, and microspheres. Liposomes are microscopic vesicles having a lipid wall comprising a lipid bilayer, and can be used as drug delivery systems herein as well. Generally, liposome formulations are preferred for poorly soluble or insoluble pharmaceutical agents. Liposomal preparations for use in the instant invention include cationic (positively charged), anionic (negatively charged) and neutral preparations. Cationic liposomes are readily available. For example, N[1-2,3-dioleyloxy)propyl]-N,N,N-triethyl-ammonium (DOTMA) liposomes are available under the tradename Lipofectin™ (GIBCO BRL, Grand Island, N.Y.). Similarly, anionic and neutral liposomes are readily available as well, e.g., from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with DOTMA in appropriate ratios. Methods for making liposomes using these materials are well known in the art.

Micelles are known in the art as comprised of surfactant molecules arranged so that their polar headgroups form an outer spherical shell, while the hydrophobic, hydrocarbon chains are oriented towards the center of the sphere, forming a core. Micelles form in an aqueous solution containing surfactant at a high enough concentration so that micelles naturally result. Surfactants useful for forming micelles include, but are not limited to, potassium laurate, sodium octane sulfonate, sodium decane sulfonate, sodium dodecane sulfonate, sodium lauryl sulfate, docusate sodium, decyltrimethylammonium bromide, dodecyltrimethylammonium bromide, tetradecyltrimethylammonium bromide, tetradecyltrimethyl-ammonium chloride, dodecylammonium chloride, polyoxyl 8 dodecyl ether, polyoxyl 12 dodecyl ether, nonoxynol 10 and nonoxynol 30. Micelle formulations can be used in conjunction with the present invention either by incorporation into the reservoir of a topical or transdermal delivery system, or into a formulation to be applied to the body surface.

Microspheres, similarly, may be incorporated into the present formulations and drug delivery systems. Like liposomes and micelles, microspheres essentially encapsulate a drug or drug-containing formulation. They are generally although not necessarily formed from lipids, preferably charged lipids such as phospholipids. Preparation of lipidic microspheres is well known in the art and described in the pertinent texts and literature.

Various additives, known to those skilled in the art, may be included in the topical formulations. For example, solvents, including relatively small amounts of alcohol, may be used to solubilize certain drug substances. Other optional additives include opacifiers, antioxidants, fragrance, colorant, gelling agents, thickening agents, stabilizers, surfactants and the like. Other agents may also be added, such as antimicrobial agents, to prevent spoilage upon storage, i.e., to inhibit growth of microbes such as yeasts and molds. Suitable antimicrobial agents are typically selected from the group consisting of the methyl and propyl esters of p-hydroxybenzoic acid (i.e., methyl and propyl paraben), sodium benzoate, sorbic acid, imidurea, and combinations thereof.

For those drugs having an unusually low rate of permeation through the skin or mucosal tissue, it may be desirable to include a second permeation enhancer in the formulation in addition to the hydroxide-releasing agent, although in a preferred embodiment the hydroxide-releasing agent is administered without any other permeation enhancers. Any other enhancers should, like the hydroxide-releasing agent itself, minimize the possibility of skin damage, irritation, and systemic toxicity. Examples of suitable secondary enhancers (or “co-enhancers”) include, but are not limited to, ethers such as diethylene glycol monoethyl ether (available commercially as Transcutol) and diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80) and lecithin (U.S. Pat. No. 4,783,450; see also); alcohols such as ethanol, propanol, octanol, benzyl alcohol, and the like; fatty acids such as lauric acid, oleic acid and valeric acid; fatty acid esters such as isopropyl myristate, isopropyl palmitate, methylpropionate, and ethyl oleate; polyols and esters thereof such as polyethylene glycol, and polyethylene glycol monolaurate (PEGML; see, e.g., U.S. Pat. No. 4,568,343); amides and other nitrogenous compounds such as urea, dimethylacetamide (DMA), dimethylformamide (DMF), 2-pyrrolidone, 1-methyl-2-pyrrolidone, ethanolamine, diethanolamine and triethanolamine; terpenes; alkanones; and organic acids, particularly citric acid and succinic acid. Azone and sulfoxides such as DMSO and N-decylmethyl sulfoxide (C10MSO) may also be used, but are less preferred. As noted earlier herein, Percutaneous Penetration Enhancers, eds. Smith et al. (CRC Press, 1995) provides an excellent overview of the field and further information concerning possible secondary enhancers for use in conjunction with the present invention.

The formulation may also contain irritation-mitigating additives to minimize or eliminate the possibility of skin irritation or skin damage resulting from the drug, the enhancer, or other components of the formulation. Suitable irritation-mitigating additives include, for example: alpha.-tocopherol; monoamine oxidase inhibitors, particularly phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; and chloroquine. The irritant-mitigating additive, if present, may be incorporated into the present formulations at a concentration effective to mitigate irritation or skin damage, typically representing not more than about 20 wt. %, more typically not more than about 5 wt. %, of the formulations.

The concentration of the active agent in the formulation can vary a great deal, and will depend on a variety of factors, including the disease or condition to be treated, the nature and activity of the active agent, the desired effect, possible adverse reactions, the ability and speed of the active agent to reach its intended target, and other factors within the particular knowledge of the patient and physician. Preferred formulations will typically contain on the order of about 0.5 wt. % to 50 wt. %, optimally about 10 wt. % to 30 wt. %, active agent.

An alternative and preferred method involves the use of a drug delivery system, e.g., a topical or transdermal “patch,” wherein the active agent is contained within a laminated structure that is to be affixed to the skin. In such a structure, the drug composition is contained in a layer, or “reservoir,” underlying an upper backing layer. The laminated structure may contain a single reservoir, or it may contain multiple reservoirs.

In one embodiment, the reservoir comprises a polymeric matrix of a pharmaceutically acceptable adhesive material that serves to affix the system to the skin during drug delivery; typically, the adhesive material is a pressure-sensitive adhesive (PSA) that is suitable for long-term skin contact, and which should be physically and chemically compatible with the active agent, hydroxide-releasing agent, and any carriers, vehicles or other additives that are present. Examples of suitable adhesive materials include, but are not limited to, the following: polyethylenes; polysiloxanes; polyisobutylenes; polyacrylates; polyacrylamides; polyurethanes; plasticized ethylene-vinyl acetate copolymers; and tacky rubbers such as polyisobutene, polybutadiene, polystyrene-isoprene copolymers, polystyrene-butadiene copolymers, and neoprene (polychloroprene). Preferred adhesives are polyisobutylenes.

The backing layer functions as the primary structural element of the transdermal system and provides the device with flexibility and, preferably, occlusivity. The material used for the backing layer should be inert and incapable of absorbing drug, hydroxide-releasing agent or components of the formulation contained within the device. The backing is preferably comprised of a flexible elastomeric material that serves as a protective covering to prevent loss of drug and/or vehicle via transmission through the upper surface of the patch, and will preferably impart a degree of occlusivity to the system, such that the area of the body surface covered by the patch becomes hydrated during use. The material used for the backing layer should permit the device to follow the contours of the skin and be worn comfortably on areas of skin such as at joints or other points of flexure, that are normally subjected to mechanical strain with little or no likelihood of the device disengaging from the skin due to differences in the flexibility or resiliency of the skin and the device. The materials used as the backing layer are either occlusive or permeable, as noted above, although occlusive backings are preferred, and are generally derived from synthetic-polymers (e.g., polyester, polyethylene, polypropylene, polyurethane, polyvinylidine chloride, and polyether amide), natural polymers (e.g., cellulosic materials), or macroporous woven and nonwoven materials.

During storage and prior to use, the laminated structure includes a release liner. Immediately prior to use, this layer is removed from the device so that the system may be affixed to the skin. The release liner should be made from a drug/vehicle impermeable material, and is a disposable element which serves only to protect the device prior to application. Typically, the release liner is formed from a material impermeable to the pharmacologically active agent and the hydroxide-releasing agent, and which is easily stripped from the transdermal patch prior to use.

In an alternative embodiment, the drug-containing reservoir and skin contact adhesive are present as separate and distinct layers, with the adhesive underlying the reservoir. In such a case, the reservoir may be a polymeric matrix as described above. Alternatively, the reservoir may be comprised of a liquid or semisolid formulation contained in a closed compartment or “pouch,” or it may be a hydrogel reservoir, or may take some other form. Hydrogel reservoirs are particularly preferred herein. As will be appreciated by those skilled in the art, hydrogels are macromolecular networks that absorb water and thus swell but do not dissolve in water. That is, hydrogels contain hydrophilic functional groups that provide for water absorption, but the hydrogels are comprised of crosslinked polymers that give rise to aqueous insolubility. Generally, then, hydrogels are comprised of crosslinked hydrophilic polymers such as a polyurethane, a polyvinyl alcohol, a polyacrylic acid, a polyoxyethylene, a polyvinylpyrrolidone, a poly(hydroxyethyl methacrylate) (poly(HEMA)), or a copolymer or mixture thereof. Particularly preferred hydrophilic polymers are copolymers of HEMA and polyvinylpyrrolidone.

Additional layers, e.g., intermediate fabric layers and/or rate-controlling membranes, may also be present in any of these drug delivery systems. Fabric layers may be used to facilitate fabrication of the device, while a rate-controlling membrane may be used to control the rate at which a component permeates out of the device. The component may be a drug, a hydroxide-releasing agent, an additional enhancer, or some other component contained in the drug delivery system.

A rate-controlling membrane, if present, will be included in the system on the skin side of one or more of the drug reservoirs. The materials used to form such a membrane are selected to limit the flux of one or more components contained in the drug formulation. Representative materials useful for forming rate-controlling membranes include polyolefins such as polyethylene and polypropylene, polyamides, polyesters, ethylene-ethacrylate copolymer, ethylene-vinyl acetate copolymer, ethylene-vinyl methylacetate copolymer, ethylene-vinyl ethylacetate copolymer, ethylene-vinyl propylacetate copolymer, polyisoprene, polyacrylonitrile, ethylene-propylene copolymer, and the like.

Generally, the underlying surface of the transdermal device, i.e., the skin contact area, has an area in the range of about 5 cm² to 200 cm², preferably 5 cm² to 100 cm², more preferably 20 cm² to 60 cm². That area will vary, of course, with the amount of drug to be delivered and the flux of the drug through the body surface. Larger patches will necessary to accommodate larger quantities of drug, while smaller patches can be used for smaller quantities of drug and/or drugs that exhibit a relatively high permeation rate.

Such drug delivery systems may be fabricated using conventional coating and laminating techniques known in the art. For example, adhesive matrix systems can be prepared by casting a fluid admixture of adhesive, drug and vehicle onto the backing layer, followed by lamination of the release liner. Similarly, the adhesive mixture may be cast onto the release liner, followed by lamination of the backing layer. Alternatively, the drug reservoir may be prepared in the absence of drug or excipient, and then loaded by “soaking” in a drug/vehicle mixture. In general, transdermal systems of the invention are fabricated by solvent evaporation, film casting, melt extrusion, thin film lamination, die cutting, or the like. The hydroxide-releasing agent will generally be incorporated into the device during patch manufacture rather than subsequent to preparation of the device. Thus, for acid addition salts of basic drugs (e.g., hydrochloride salts of amine drugs, such as phenylpropanolamine hydrochloride), the hydroxide-releasing agent will neutralize the drug during manufacture of the drug delivery system, resulting in a final drug delivery system in which the drug is present in nonionized, neutral form along with an excess of hydroxide-releasing agent to serve as a permeation enhancer. For nonionized acidic drugs, the hydroxide-releasing agent will neutralize such drugs by converting them to the ionized drug in salt form.

In a preferred delivery system, an adhesive overlayer that also serves as a backing for the delivery system is used to better secure the patch to the body surface. This overlayer is sized such that it extends beyond the drug reservoir so that adhesive on the overlayer comes into contact with the body surface. The overlayer is useful because the adhesive/drug reservoir layer may lose its adhesion a few hours after application due to hydration. By incorporating such adhesive overlayer, the delivery system remains in place for the required period of time.

Other types and configurations of transdermal drug delivery systems may also be used in conjunction with the method of the present invention, i.e., the use of a hydroxide-releasing agent as a permeation enhancer, as will be appreciated by those skilled in the art of transdermal drug delivery. See, for example, Ghosh, Transdermal and Topical Drug Delivery Systems (Interpharm Press, 1997), particularly Chapters 2 and 8. As with the topically applied formulations of the invention, the composition containing drug and hydroxide-releasing agent within the drug reservoir(s) of these laminated system may contain a number of components. In some cases, the drug and hydroxide-releasing agent may be delivered “neat,” i.e., in the absence of additional liquid. In most cases, however, the drug will be dissolved, dispersed or suspended in a suitable pharmaceutically acceptable vehicle, typically a solvent or gel. Other components that may be present include preservatives, stabilizers, surfactants, and the like.

The invention accordingly provides a novel and highly effective means for increasing the flux of an active agent through the body surface (skin or mucosal tissue) of a human or animal. The hydroxide-releasing agents discussed herein, employed in specific amounts relative to a formulation or drug reservoir, may be used as permeation enhancers with a wide variety of drugs and drug types, including free acids, free bases, acid addition salts of basic drugs, basic addition salts of acidic drugs, nonionizable drugs, peptides and proteins. Surprisingly, the increase in permeation is not accompanied by any noticeable tissue damage, irritation, or sensitization. The invention thus represents an important advance in the field of drug delivery.

It is to be understood that while the invention has been described in conjunction with the preferred specific embodiments thereof, the foregoing description is intended to illustrate and not limit the scope of the invention. Other aspects, advantages and modifications will be apparent to those skilled in the art to which the invention pertains. Furthermore, the practice of the present invention will employ, unless otherwise indicated, conventional techniques of drug formulation, particularly topical and transdermal drug formulation, which are within the skill of the art. Such techniques are fully explained in the literature. See Remington: The Science and Practice of Pharmacy, cited supra, as well as Goodman & Gilman's The Pharmacological Basis of Therapeutics, 9th Ed. (New York: McGraw-Hill, 1996).

The attached sheets exemplify both formulations and results of experiments that involve both the method and composition of this invention.

The present invention provides a method and related composition for enhancing the rate at which an active agent comprising a PPI can be administered in stable form to a patient's body surface in order to permeate into and/or through the body surface, including a PPI in combination with a hydroxide-releasing agent as a permeation enhancer, and a carrier selected from the group of alkyl and alkylene diols and glycols.

What is claimed is:

1. A method for enhancing the rate at which an active agent comprising a proton pump inhibitor can be administered in stable form to a patient's body surface in order to permeate into and/or through the body surface, the method comprising the use of a hydroxide-releasing agent as a permeation enhancer to increase the flux of the proton pump inhibitor through a patient's skin or mucosal tissue.

2. A method according to claim 1 wherein the amount of hydroxide-releasing agent employed is optimized to enhance permeation while minimizing or eliminating the possibility of skin damage, irritation or sensitization.

3. A method according to claim 1 wherein the hydroxide releasing agent and proton pump inhibitor are used in combination with a carrier selected from the group consisting of alkyl diols, alkylene diols, and glycols, in order to both solubilize and stabilize the proton pump inhibitor for its intended use.

4. A method according to claim 3 wherein the composition provides an optimal combination of both proton pump inhibitor stability and permeation.

5. A method according to claim 4, wherein the composition provides therapeutic levels to the body while meeting or exceeding the stability requirement that the compositions show no significant change during 6 months of storage under conditions of accelerated testing at 40 (+/−2) degrees C., 75% (+/−5%) relative humidity.

6. A method according to claim 1 wherein the proton pump inhibitor is a substituted pyridyl methylsulfinyl benzimidazole selected from the group consisting of omeprazole, lansoprazole, esomeprazole, pantoprazole and raberprazole.

7. A method according to claim 6 wherein the proton pump inhibitor is present at a concentration of between about 5 to about 30% by weight of the composition.

8. A method according to claim 3 wherein the carrier is selected from the group consisting of 1,3-butanediol, dipropylene glycol, and hexylene glycol.

9. A method according to claim 8 wherein the carrier is present in an amount of between about 5 to about 40% by weight, based on the weight of the composition.

10. A method for enhancing the rate at which an active agent comprising a proton pump inhibitor can be administered in stable form to a patient's body surface in order to permeate into and/or through the body surface, the method comprising the use of a hydroxide-releasing agent as a permeation enhancer to increase the flux of the proton pump inhibitor through a patient's skin or mucosal tissue,
wherein the amount of hydroxide-releasing agent employed is optimized to enhance permeation while minimizing or eliminating the possibility of skin damage, irritation or sensitization, and
wherein the hydroxide releasing agent and proton pump inhibitor are used in combination with a carrier selected from the group consisting of 1,3-butanediol, dipropylene glycol, and hexylene glycol, the carrier being present in an amount of between about 5 to about 40% by weight, based on the weight of the composition, and
wherein the composition provides therapeutic levels to the body while meeting or exceeding the stability requirement that the compositions show no significant change during 6 months of storage under conditions of accelerated testing at 40 (+/−2) degrees C., 75% (+/−5%) relative humidity, and
wherein the proton pump inhibitor is a substituted pyridyl methylsulfinyl benzimidazole selected from the group consisting of omeprazole, lansoprazole, esomeprazole, pantoprazole and raberprazole present at a concentration of between about 5 to about 30% by weight of the composition.

11. A composition suitable for enhancing the rate at which an active agent comprising a proton pump inhibitor can be administered in stable form to a patient's body surface in order to permeate into and/or through the body surface, the composition comprising the combination of proton pump inhibitor and a hydroxide-releasing agent as a permeation enhancer to increase the flux of the proton pump inhibitor through a patient's skin or mucosal tissue.

12. A composition according to claim 11 wherein the amount of hydroxide-releasing agent employed is optimized to enhance permeation while minimizing or eliminating the possibility of skin damage, irritation or sensitization.

13. A composition according to claim 11 wherein the composition further comprises a carrier selected from the group consisting of alkyl diols, alkylene diols, and glycols, in order to both solubilize and stabilize the proton pump inhibitor for its intended use.

14. A composition according to claim 13 wherein the composition provides an optimal combination of both proton pump inhibitor stability and permeation.

15. A composition according to claim 14, wherein the composition provides therapeutic levels to the body while meeting or exceeding the stability requirement that the compositions show no significant change during 6 months of storage under conditions of accelerated testing at 40 (+/−2) degrees C., 75% (+/−5%) relative humidity.

16. A composition according to claim 11 wherein the proton pump inhibitor is a substituted pyridyl methylsulfinyl benzimidazole selected from the group consisting of omeprazole, lansoprazole, esomeprazole, pantoprazole and raberprazole.

17. A composition according to claim 16 wherein the proton pump inhibitor is present at a concentration of between about 5 to about 30% by weight of the composition.

18. A composition according to claim 13 wherein the carrier is selected from the group consisting of 1,3-butanediol, dipropylene glycol, and hexylene glycol.

19. A composition according to claim 18 wherein the carrier is present in an amount of between about 5 to about 40% by weight, based on the weight of the composition.

20. A composition for enhancing the rate at which an active agent comprising a proton pump inhibitor can be administered in stable form to a patient's body surface in order to permeate into and/or through the body surface, the composition comprising a proton pump inhibitor in combination with a hydroxide-releasing agent as a permeation enhancer to increase the flux of the proton pump inhibitor through a patient's skin or mucosal tissue,
wherein the amount of hydroxide-releasing agent employed is optimized to enhance permeation while minimizing or eliminating the possibility of skin damage, irritation or sensitization, and
wherein the hydroxide releasing agent and proton pump inhibitor are used in combination with a carrier selected from the group consisting of 1,3-butanediol, dipropylene glycol, and hexylene glycol, the carrier being present in an amount of between about 5 to about 40% by weight, based on the weight of the composition, and
wherein the composition provides therapeutic levels to the body while meeting or exceeding the stability requirement that the compositions show no significant change during 6 months of storage under conditions of accelerated testing at 40 (+/−2) degrees C., 75% (+/−5%) relative humidity, and
wherein the proton pump inhibitor is a substituted pyridyl methylsulfinyl benzimidazole selected from the group consisting of omeprazole, lansoprazole, esomeprazole, pantoprazole and raberprazole present at a concentration of between about 5 to about 30% by weight of the composition.
